(19) United States
(12) Patent Application Publication
(10) Pub. No.: US 2003/0170194 A1
(43) Pub. Date: Sep. 11, 2003

(54) Pharmaceutical and/or cosmetic composition containing an organosiloxane and a phospholipid

(76) Inventors: Ralf Piotrowiak, Bad Oeynhausen (DE); Bernd G Siegfried, Limburgerhof (DE)

Correspondence Address: GREENBERG TRAURIG, P.C., 77 WEST WACKER DRIVE, CHICAGO, IL, 60601-1732, US

(21) Appl. No.: 10/276,766

(22) PCT Filed: Apr. 14, 2001
(86) PCT No.: PCT/DE01/01483

(30) Foreign Application Priority Data
May 19, 2000 (DE) 100 24 413.0

(51) Int. Cl. A61K 7/06; A61K 7/11; A61K 7/075; A61K 7/08

(52) U.S. Cl. 424/70.12; 424/70.21

(57) ABSTRACT

A pharmaceutical and/or cosmetic composition for use in humans, animals or plants is described, comprising at least one pharmaceutical and/or cosmetic active ingredient and at least one organosilicon compound based on oligomeric and/or polymeric diorganosiloxane, whereby the composition also contains at least one phospholipid.

The present invention relates to a pharmaceutical and/or cosmetic composition for use in humans, animals or plants having the features of the preamble of patent claim 1.

Cosmetic and/or pharmaceutical compositions of the type defined in the preamble are known in various formulations and forms of administration. These known compositions have at least one corresponding pharmaceutical and/or cosmetic active ingredient, while other ingredients of the known compositions are used to make it possible to obtain certain desired properties of the active ingredient itself or the composition, e.g., a specific form of administration.

There are also known pharmaceutical compositions for use in humans, where these known compositions have at least one organosilicon compound based on an oligomeric and/or polymeric diorganosiloxane in addition to at least one pharmaceutical active ingredient. For example, U.S. Pat. No. 5,582,815 describes a therapeutic or cosmetic composition which is applied topically in the form of an aerosol and which contains a high concentration of a polydiorganosiloxane in addition to a therapeutic or cosmetic active ingredient, preferably an antimycotic or antifungal ingredient.

In many cases, however, there is the problem that corresponding pharmaceutical and/or cosmetic active ingredients have little or no solubility or dispersibility in the particular diorganosiloxane used, so that producing a corresponding pharmaceutical and/or cosmetic composition that is stable with respect to separation poses considerable problems.

The object of the present invention is to make available a pharmaceutical and/or cosmetic composition for use on humans, animals or plants, such that it will have a particularly high stability.

This object is achieved according to this invention by a pharmaceutical and/or cosmetic composition of the type defined in the preamble having the characterizing feature of patent claim 1.

The pharmaceutical and/or cosmetic composition according to this invention for use in humans, animals or plants comprises at least one pharmaceutical and/or cosmetic active ingredient and at least one organosilicon compound based on an oligomeric and/or polymeric diorganosiloxane, whereby the composition according to this invention also contains at least one phospholipid. In other words, the composition according to this invention differs from the known composition discussed in conjunction with U.S. Pat. No. 5,582,815 in that the inventive composition additionally contains at least one phospholipid.

The term active pharmaceutical ingredient as used with regard to the inventive composition is also intended to include active ingredients which have a certain biological activity in plants, as explained below.

The composition according to this invention described above has a number of advantages. It should first be pointed out that it has an excellent stability, so there no separation of ingredients and no agglomeration of the active ingredient respectively active ingredients, nor is there any unwanted chemical degradation of the active ingredient or the active ingredients even after prolonged storage, in particular even at elevated temperatures. This is all the more surprising, since it is known that phospholipids, in particular phospholipids that contain phosphatidylcholine, tend to undergo an unwanted chemical degradation during storage, and such degradation products can then entail the risk of catalyzing chemical degradation of the active ingredient respectively the active ingredients. However, it has surprisingly been found that this does not occur in the inventive composition as described above.

In addition, it has been found that due to the presence of the at least one phospholipid in the inventive composition, the solubility and/or dispersibility of the active ingredient respectively ingredients is made possible at all or at least is greatly improved in many cases, so that the possibilities of producing formulations are expanded to a plurality of active ingredients in comparison with such known compositions containing only the above-mentioned organosilicon compound in addition to the active ingredient.

Thus, for example, it has been found that the solubility of the active ingredient clotrimazole in hexamethyldisiloxane alone is less than 0.025% by weight, while this active ingredient will dissolve in a 10-fold higher concentration in a composition containing 10% by weight phospholipid and 90% by weight hexamethyldisiloxane, whereby the phospholipid used for this purpose comprises 25% by weight ethanol and 75% by weight phospholipid, which in turn contains 76±3% by weight phosphatidylcholine, 3±3% by weight lysophosphatidylcholine, up to 8% by weight phosphatidic acid, up to 4% by weight phosphatidylethanolamine and a maximum of 9% by weight other phospholipid and non-phospholipid constituents, such as in particular oils, fats and/or triglycerides.

In addition, addition of the at least one phospholipid to the inventive composition causes the composition, when applied topically, to penetrate directly within an extremely short period of time into the skin of human or animal or into the external plant layers of plants treated with it, so that such topically applied compositions according to this invention cannot be rubbed off inadvertently or cannot be washed off unintentionally. With such an use, a gelatinous formulation develops on the skin within a few seconds, so that the composition, which is originally liquid when applied, adheres to the zone where it is applied because of this change in consistency, and then is rapidly absorbed into the interior of the body.

Furthermore, the inventive composition has a moisturizing effect to provide care for external areas of the body treated with it accordingly, with the result that a corresponding healing or improvement is induced in an accelerated manner. Skin irritation such, as that observed in particular in sensitive human and animal users with the use of known compositions which do not contain any phospholipid, does not occur when using the composition according to this invention. Therefore, the inventive composition, which contains a maximum of 14% by weight alcohol, does not cause any additional pain when applied topically to sensitive or predamaged skin, although this is usually the case with conventional topical formulations containing more than 14.5% by weight alcohol, so that the corresponding user need not overcome the resulting obstacle to use in the case of the composition according to this invention, thus ensuring steady and reproducible use which ultimately ensures the success of the therapeutic treatment. In addition, due to the at least one phospholipid additive in the composition according to this invention, in the case of a plurality of differently structured active ingredients it is ensured that transparent solutions and/or transparent, finely dispersed systems are obtained, which can be sterile filtered on the one hand, while on the other hand being inspected easily for foreign particles due to their transparency. This possibility of inspection is very important if the inventive composition is formulated to be administered as an infusion or injection.

In particular for such embodiments of the inventive composition which are used topically in humans or animals, such an organosilicon compound of the type defined in the preamble is contained in the composition, its vapor pressure being between 1 kPa and 7 kPa, preferably between 3.5 kPa and 5.7 kPa, and its boiling point varying between 15° C. and 150° C. The values given above for the vapor pressure are based on a temperature of 25° C., while the boiling point values are based on normal pressure. These embodiments of the composition according to this invention have the particular advantage that they are absorbed internally very rapidly, preferably between 2 seconds and 30 seconds after being applied to the respective body areas, so that no residues and especially no greasy residues, remain on the surface. Therefore, this embodiment of the inventive composition ensures to a high degree that the quantity applied topically will in fact enter the body within the shortest possible period of time and will prevent soiling of the user's clothing, thus providing a high user friendliness of the composition according to this invention.

Another advantage of the inventive composition which contains the above-mentioned organosilicon compounds is that such organosilicon compounds produce a cooling effect in the area of the composition applied according to this invention because they evaporate rapidly, and this cooling effect is perceived as very pleasant, especially when the inventive composition is applied topically for treatment of inflammatory conditions or contusions or for treatment of other sports injuries.

It is especially suitable if the composition according to this invention contains a cyclic oligomeric siloxane compound of general formula I as the organosilicon compound

(R₁—SiO—R₂)ₙ Formula I

and/or a linear siloxane compound of general formula II

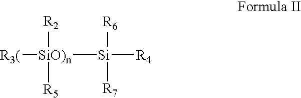

whereby in the two formulas I and II

R₁, R₂, R₃, R₄, R₅, R₆ and/or R₇ may each be the same or different and denote a C₁ to C₄-alkyl group, preferably a saturated C₁ to C₄-alkyl group, and/or

R₃ and/or R₄, R₆ and/or R₇ may be the same or different and denote hydrogen and/or a hydroxyl group and

n is an integer between 0 and 30, preferably between 0 and 8.

Such siloxanes, which are also known as volatile silicones and are commercially available, have a low surface tension, preferably a surface tension in the range between 18 dyn/cm and 21 dyn/cm, are inert and have a high wetting power, so they are especially preferred in formulations that are applied topically. This is not only related to the fact that, because of their inertness, these special cyclic and/or linear siloxane compounds impart the required stability to a composition formulated with them, but it is also related to the fact that they evaporate very soon after being applied and also have a good cleansing and dissolving power for the fats and/or fat-like substances and other impurities present on the skin surface, so that a cleansing effect also occurs when they are applied and optionally rubbed in lightly. In addition, these cyclic and/or linear siloxanes are water repellent and are tolerated well on the skin, so they are also preferred in particular for topical application.

In particular, the inventive composition contains

the cyclic tetramer of general formula I as the organosilicon compound in especially preferred embodiments, whereby in formula I, n is 4, and R₁ and R₂ each denote a CH₃-group,

the cyclic pentamer of general formula I, whereby in formula I, n stands for 5, and R₁ and R₂ each denote a CH₃-group and/or

a linear siloxane of general formula II, whereby in formula II, n stands for 2, 3 or 4, and R₂ to R₇ each denote a CH₃-group, whereby hexamethyldisiloxane is especially preferred.

The inventive composition may of course also contain a mixture of the siloxanes described above.

With regard to the at least one phospholipid contained in the composition according to this invention, it should be pointed out that this is preferably a phospholipid or phospholipid mixture isolated from natural starting materials, in particular soybean, safflower, cottonseed, sunflower seed and/or egg, whereby such phospholipids are preferably used in the form of oil-free phospholipids.

Especially good results with regard to stability and the advantageous properties described above for the composition according to this invention—e.g., a high dissolving power and/or dispersing power, sterile filterability, the ease with which it is possible to inspect for foreign particles and the moisturizing effects—are achieved with such embodiments of the composition according to this invention in which the phospholipid present is at least one phospholipid having a high phosphatidylcholine content (1,2-diacylglycero-3-phosphatidylcholine), whereby such phospholipids containing more than 70% by weight, in particular at least 80% by weight and preferably more than 90% by weight phosphatidylcholine, based on the total weight of the particular phospholipid used, are preferably selected.

If in such a phospholipid or phospholipid mixture having a high phosphatidylcholine content, its acyl groups in positions 1 and 2 additionally consist of 10% by weight to 15% by weight of the palmitic acid group, 1.4% by weight to 4% by weight of the stearic acid group, 3% by weight to 10% by weight of the oleic acid group, 61% by weight to 71% by weight of the linoleic acid group and 3% by weight to 7% by weight of the linolenic acid group, based on the sum of all acyl groups in positions 1 and 2, then such a phosphatidylcholine in combination with the organosilicon compounds described above imparts a high chemical stability to the composition according to this invention, so that unwanted degradation of the active ingredient or the active ingredient mixture does not occur at all or occurs only to a completely subordinate extent even after several months of storage under extreme conditions.

In another embodiment of the composition according to this invention, it contains a hydrogenated phospholipid as the phospholipid, preferably a hydrogenated phosphatidylcholine. Such hydrogenated phospholipids also facilitate the production of transparent compositions, so that only a brief mixing of ingredients of the inventive composition is necessary here to prepare suitably sterile-filterable and transparent solutions and/or fine dispersions of the active ingredient which can then also be administered reliably by injection or infusion accordingly, optionally after sterile filtration through a 0.2 μm filter and/or after additional sterilization.

In general, the ratio of the organosilicon compound to the at least one phospholipid depends in particular on the desired form of administration, the organosilicon compound selected, the particular phospholipid used and the at least one pharmaceutical and/or cosmetic active ingredient contained in the inventive composition. Excellent dissolving and dispersing properties combined with an excellent stability can be achieved by the fact that the composition according to this invention contains the organosilicon compound in a concentration between 5% by weight and 96% by weight, and the at least one phospholipid is present in a concentration between 2% by weight and 30% by weight, each based on the total weight of the composition. It is especially suitable if the composition according to this invention contains between 20% by weight and 85% by weight of the organosilicon compound and between 5% by weight and 20% by weight of the at least one phospholipid.

By varying and coordinating the organosilicon compound described above and the at least one phospholipid described above in combination with water, it is also possible to formulate embodiments of the composition according to this invention in which the water content is varied between 0% by weight and 5% by weight and in which the organosilicon compound contained in the composition and the phospholipid have been blended together to the extent that the composition will spontaneously develop liposomes and/or micelles on coming in contact with water. It has been found that the slight water concentration occurring naturally in the skin and in particular in the upper strata of skin is surprisingly sufficient to spontaneously form these liposomes and/or micelles on the skin surface, so that such a refinement of the composition according to this invention is especially preferred for topical application.

Preferably the organosilicon compounds, the phospholipid and the water are coordinated with one another in the embodiment of the composition according to this invention, as described above, so that the liposomes and/or micelles which are formed spontaneously when water is added will have an average particle size between 50 nm and 4000 nm, in particular between 250 nm and 1500 nm, so that such liposomes and/or micelles are especially suitable for transporting the active ingredients contained in the inventive composition rapidly and completely into the interior of the human or animal body or into the interior of the plant after topical application. This also ensures in particular that the respective active ingredient will rapidly and without destruction reach the area where it should have the cosmetic and/or pharmaceutical efficacy in this regard.

Depending on the particular use of the composition according to the invention and its form of administration, the inventive composition optionally also contains water, alcohol, antioxidants, non-phospholipid emulsifiers, pharmaceutical active ingredients, cosmetic active ingredients and/or other additives such as gel-forming substances, thickeners, perfumes, preservatives or the like, in addition to the phospholipid and the at least one organosilicon compound of the type described in the preamble.

In particular with the composition according to this invention, the concentration of the at least one pharmaceutical active ingredient and/or the at least one cosmetic active ingredient in the composition is varied between 0.001% by weight and 30% by weight, preferably between 0.1% by weight and 15% by weight, each based on the ready-to-use composition.

An especially suitable embodiment of the composition according to this invention, which is preferably used for topical application, provides for the composition to also contain at least one alcohol, in particular ethanol, 1-propanol and/or 2-propanol, where the concentration of the at least one alcohol amounts to a maximum of 14% by weight and is preferably varied between 0.1% by weight and 8.5% by weight, each based on the ready-to-use composition. This embodiment of the composition according to this invention has the additional advantage due to the alcohol content that this embodiment of the inventive composition is already sterile from the beginning, so that the use of additional preservatives may be omitted. In addition, it has surprisingly been found that even when the particular user has a high sensitivity to alcohol, there is no skin irritation if the alcohol concentration in the composition according to this invention is limited to a maximum of 14% by weight. This is attributed to the fact that the composition according to this invention contains as essential ingredients the combination of organosilicon compound with phospholipid, so that the negative effects of alcohol on the skin are precluded due to this combination, which is gentle on the skin and also has a curative effect on the skin. Even if such an embodiment of the inventive composition is applied topically to highly inflamed skin areas or to highly inflamed areas of the mucous membranes, no negative side effects such as an unpleasant and painful burning could be detected.

With regard to the at least one active ingredient contained in the inventive composition, it should be pointed out that first the choice of active ingredient depends on whether the composition according to this invention is to be used on humans, animals or plants.

For human application or for use in animals, the inventive composition contains in particular a pharmaceutical active ingredient, which is selected from the group consisting of local anesthetics, antiallergics, dermatologics, active ingredients to combat influenza infections and colds, active ingredients for treatment of neuropathies, active ingredients for treatment of circulation disorders, chemotherapeutics, quinine, antimycotics, antibiotics, thalidomide, analgesics, non-steroidal anti-inflammatory drugs, leukotrienes, leukotriene inhibitors, antiandrogens, corticosteroids, opiate receptor antagonists, blood coagulation inhibitors, platelet aggregation inhibitors, histamine antagonists, regulatory and enzymatic peptides and proteins, nucleic acids and oligopeptides, antidiabetics, prostaglandins, prostaglandin synthesis inhibitors, antiviral or virustatic substances, antimicrobial substances, active agents against prions, immunosuppressants, hormones, active ingredients for treatment of wounds and chronic wounds in particular, vitamins, plant extracts or extracts of plant extracts, psychopharmaceuticals, active ingredients to influence sleep, analeptics, general anesthetics, muscle relaxants, antiepileptics, antiparkinsonism drugs, antiemetics, substances that act on the ganglia, substances that act on the sympathetic nervous system, substances that act the parasympathetic nervous system, calcium antagonists, cardiovascular agents, antiasthmatics, antitussives, expectorants, hepatics, diuretics, choleretics, disinfectants, trace elements, anti-infectives, cytostatics, antimetabolites, hormone antagonists, immunomodulators and/or active ingredients against internal or external parasites.

If, however, the composition according to this invention is used in the area of plants, then in this special embodiment of the composition according to this invention, the active ingredient may be a fungicide, an insecticide, an herbicide and/or a fertilizer that can be absorbed through leaf parts and/or above-ground parts of the plant.

As emphasized above repeatedly, the composition according to this invention is preferably also applied topically, whereby all possible topical forms of administration, e.g., topical administration as a gel, cream, spray, powder, or plaster or patch are also conceivable for this purpose.

In particular, the composition according to this invention for topical application has a liquid to pasty viscosity, the viscosity of the composition preferably being varied between 0.4 cSt and 10,000 cSt, in particular between 0.6 cSt and 1500 cSt. The viscosity data used in the present patent application are all determined at 25° C. This liquid to pasty form of administration of the inventive composition is very easy to apply on the one hand, while on the other hand being absorbed very rapidly into the interior of the body so that ease of handling and the efficacy of the composition according to this invention are ensured.

In particular with the topical application of the composition according to this invention as described above, the at least one organosilicon compound and the at least one phospholipid plus optionally a solvent, preferably an alcohol, are coordinated with regard to their weight ratios, so that after applying the composition to human or animal skin in a concentration between 0.05 g and 0.3 g per 100 cm² skin, the composition thus applied is absorbed within a period of 1 second to 60 seconds, preferably within a period of 3 seconds to 20 seconds, so that no visible residues remain on the skin. This then prevents soiling of the user's clothing or inadvertent removal or rubbing off of the composition thus applied topically, which thus ensures easy and reproducible use of the composition according to this invention.

In addition to or instead of the active ingredients mentioned above, another embodiment of the inventive composition contains at least one vitamin, and in particular vitamin C, vitamin E, vitamin A and/or pro-vitamin A, and these vitamins may optionally also be interpreted as pharmaceutical and/or cosmetic active ingredients.

As explained above repeatedly, the inventive composition may also be made up into any other form of administration in addition to the topical form of administration, in which case a dose of the composition may be in the form of a capsule, tablet, suppository, plaster, patch, spray, mist, gel, powder and/or cream. In addition to these specific forms of administration and/or the especially preferred topical use, the composition according to this invention may also be administered equally well by buccal, nasal, oral or anal forms of administration or by injection and/or infusion.

Advantageous refinements of the composition according to this invention are characterized in the sub-claims.

With regard to the term water as used in the present text, it should be pointed out that this term water includes not only water per se, i.e., distilled and/or deionized water, but also all other aqueous systems, e.g., in particular saline solutions or buffered solutions, preferably phosphate buffer, citrate buffer and/or acetate buffer.

Alcohol in the sense of the present invention includes all aliphatic alcohols with a carbon chain length of 1 to 30 hydroxyl groups as well as those with 1 to 10 hydroxyl groups, but preferably the low-molecular monohydric C₂ to C₅-alcohols, as well as the corresponding dihydric glycols.

The composition according to this invention is explained in greater detail below on the basis of the examples.

### EXAMPLE 1

Sprayable Gel Formulation Containing Clotrimazole as the Active Ingredient

A sprayable gel formulation was prepared, containing the following ingredients:

| | | |
|---|---|---|
| Clotrimazole | 1% | by weight |
| 2-Propanol | 8% | by weight |
| Hexamethyldisiloxane | 78.8% | by weight |
| Phospholipid | 10% | by weight |
| (75% by weight phosphatidylcholine, | | |
| 25% by weight ethanol) | | |
| Peppermint oil | 0.2% | by weight |
| 1,2-Propandiol | 2% | by weight |

To prepare this formulation, the cosmetic active ingredient mentioned above was placed first in the reactor and dissolved in the phospholipid, containing 75% by weight phosphatidylcholine and 25% by weight ethanol, with the addition of approximately half of the amount of hexamethyldisiloxane indicated above, while stirring at room temperature. After obtaining a clear solution, the remaining partial amount of hexamethyldisiloxane was added at room temperature while stirring.

The sprayable formulation prepared in this way, which had good gel-forming and film-forming properties and also had a powerful wetting effect was applied to the back of the hand from a distance of 20 cm with two spray pump doses (each 0.2 ml from a mechanical spray system, brown glass bottle sealed with a Mistette MARK II spray head). A uniformly distributed homogeneous film developed in the process within 60 seconds, it had completely evaporated or been absorbed into the skin without leaving behind a residue.

To determine the stability, the active ingredient content was measured immediately after producing. After a storage time of 60 days at 40° C. and a storage time of 120 days, also at 40° C., no loss of active ingredient could be detected. The main degradation product of clotrimazole, i.e., the carbinol, also could not be detected after a corresponding storage.

In addition, storage experiments were conducted, in which a sample of the above-mentioned spray formulation was stored for three months at 4° C., three months at 25° C. and three months at 40° C., respectively. No phase separation or crystallization occurred with any of the samples stored in this way. All these stored samples had a low viscosity, were slightly yellowish in color, transparent and sprayable, like the starting sample.

In addition, the spray formulation described above could be inspected for foreign particles, could be sterile-filtered (0.2 μm filter) and could be prepared by simple stirring without homogenization and without any additional increase in temperature or change in pressure.

To detect the pharmaceutical application of the antimycotic spray formulation having a high efficacy against all dermatological mycoses, in particular athlete's foot, vaginal mycoses and/or nail bed mycoses which are accessible to external treatment, the spray formulation described above, prepared according to example 1, was tested in comparison with a traditional commercial product which also contained 1% by weight clotrimazole as an active ingredient in a formulation containing more than 50% isopropanol and also Macrogol 400 as well as propylene glycol, a blind compatibility study (three spray pump doses of 0.2 ml each three times a day applied to approximately 100 cm² skin in the abdominal area) was conducted over a treatment period of ten days on 46 healthy volunteers (2×23 volunteers per group).

As result of this study, it can be concluded that no skin reaction was discernible in any of the volunteers treated with the spray formulation according to example 1. There was no drying effect and there were also no signs of any intolerance in the sprayed area.

Six of the volunteers who were treated with the traditional product showed definitely discernible signs of drying in the treated area of the abdominal skin in comparison with the adjacent untreated area. Three of these volunteers also showed skin irritation in the form of reddened skin and pustules in addition to the skin drying effect.

### EXAMPLE 2

Four different formulations of the acetylsalicylic acid [aspirin] active ingredient to be applied topically were prepared, and the procedure followed here to prepare these formulations was the same as that described above for example 1. The formulations in this regard had the following ingredients:

Formulation A

| | |
|---|---|
| Acetylsalicylic acid | 12.1% by weight |
| Phospholipid | 28.2% by weight |
| Isopropanol | 38.1% by weight |
| Hexamethyldisiloxane | 21.6% by weight |

Formulation B

| | | |
|---|---|---|
| Acetylsalicylic acid | 6.95% | by weight |
| Phospholipid | 16.2% | by weight |
| Isopropanol | 22.2% | by weight |
| Octamethylcyclotetrasiloxane | 54.65% | by weight |

Formulation C

| | | |
|---|---|---|
| Acetylsalicylic acid | 5.2% | by weight |
| Phospholipid | 12.1% | by weight |
| Isopropanol | 16.6% | by weight |
| Hexamethyldisiloxane | 66.1% | by weight |

Formulation D

| | | |
|---|---|---|
| Acetyl salicylic acid | 3% | by weight |
| Phospholipid | 7.2% | by weight |
| Isopropanol | 9.8% | by weight |
| Hexamethyldisiloxane | 80% | by weight |

In the above-mentioned formulas A through D, a solution of 75% by weight phosphatidylcholine in 25% by weight ethanol was used as the phospholipid.

Formulations A through B described above are used for topical and local epicutaneous application, especially in the area of pain control (headaches, muscle pains, rheumatism) and for thinning the blood. These formulations were still stable even after storage for more than three months at a storage temperature of 40° C.

### EXAMPLE 3

A sprayable and gel-forming composition for treatment of sports injuries and accident injuries, soft tissue rheumatism, pain in the motor apparatus involving the joints, muscles and/or tendons caused by various accidents, overstressing and/or other diseases was prepared from the following ingredients:

| | | |
|---|---|---|
| S-(+)-Flurbiprofen | 0.35% | by weight |
| Hexamethyldisiloxane | 60.95% | by weight |
| Phospholipid | 38.7% | by weight |

### EXAMPLE 4

A composition for dosing in hard gelatin capsules for systemic treatment of so-called banal pain (headaches, joint pain, menstrual-related pain), postoperative pain and pain caused by diseases from the rheumatic group was prepared from the following ingredients:

| | | |
|---|---|---|
| S-(+)-Flurbiprofen | 0.35% | by weight |
| Hexamethyldisiloxane | 19.65% | by weight |
| Phospholipid | 80% | by weight |

The above-mentioned composition was packaged in hard gelatin capsules (size 1) with 450 μl of the formulation given above.

### EXAMPLE 5

A composition for dosing in hard gelatin capsules for systemic treatment of so-called banal pain (headaches, joint pain, menstrual-related pain), postoperative pain and pain caused by disease from the rheumatic group was prepared from the following ingredients:

| | | |
|---|---|---|
| Dexibuprofen | 9.1% | by weight |
| Hexamethyldisiloxane | 21.2% | by weight |
| Phospholipid | 69.7% | by weight |

The composition indicated above was apportioned into doses in hard gelatin capsules (size 1) containing 450 μl of the formulation listed above.

### EXAMPLE 6

A composition for dosing in hard gelatin capsules for systemic treatment of so-called banal pain (headaches, joint pain, menstrual-related pain), postoperative pain and pain caused by disease from the rheumatic group was prepared from the following ingredients:

| | | |
|---|---|---|
| Dexibuprofen | 15% | by weight |
| Hexamethyldisiloxane | 35% | by weight |
| Phospholipid | 50% | by weight |

The composition indicated above was apportioned into doses in hard gelatin capsules (size 1) containing 450 μl of the formulation listed above.

The phospholipid used in Examples 4 through 6 contained 76±3% by weight phosphatidylcholine, 3±3% by weight lysophosphatidylcholine, up to 8% by weight phosphatidic acid, up to 4% by weight phosphatidylethanolamine and a maximum of 9% by weight other phospholipid and non-phospholipid ingredients, such as oils, fats and/or triglycerides in particular.

### EXAMPLE 7

A liquid composition for treatment of eczema, such as in particular endogenous, toxic-degenerative and seborrheic eczema, allergic contact eczema, stasis eczema, inflammatory and allergic skin conditions, psoriasis, sunburn and alopecia areata was prepared from the following ingredients:

| | | |
|---|---|---|
| Hydrocortisone, pure | 0.1% | by weight |
| Hexamethyldisiloxane | 27.5% | by weight |
| 2-Propanol, ultra-high purity | 5% | by weight |
| Phospholipid | 10.4% | by weight |
| Emusifier 10 | 2% | by weight |
| (commercial product from Dow Corning) | | |
| Water, ultra-high purity | 45% | by weight |
| Phosphate buffer 2, pH 4.8, 10-fold | 10% | by weight |

The aqueous composition indicated above is applied topically or epicutaneous or sprayed once a day (q.d.) to three times a day (t.i.d.) with one to two spray pump doses (each 0.2 ml) onto 100 cm² of the diseased skin area.

### EXAMPLE 8

A liquid composition for treatment of eczema, such as in particular endogenous, toxic-degenerative and seborrheic eczema, allergic contact eczema, stasis eczema, inflammatory and allergic skin conditions, psoriasis, sunburn and alopecia areata was prepared from the following ingredients:

| | | |
|---|---|---|
| Hydrocortisone, pure | 0.1% | by weight |
| Hexamethyldisiloxane | 65.4% | by weight |
| 2-Propanol, ultra-high purity | 5% | by weight |
| Phospholipid | 8% | by weight |
| Silmogen Carrier | 21.3% | by weight |
| (commercial product from Dow Corning) | | |
| Peppermint oil | 0.15% | by weight |

When the anhydrous sprayable composition described above was applied to the skin, it spontaneously developed liposomes which had an average particle size in the range of 194 nm.

### EXAMPLE 9

A liquid composition for treatment of eczema, such as in particular endogenous, toxic-degenerative and seborrheic eczema, allergic contact eczema, stasis eczema, inflammatory and allergic skin conditions, psoriasis, sunburn and alopecia areata was prepared from the following ingredients:

| | | |
|---|---|---|
| Hydrocortisone, pure | 0.1% | by weight |
| Hexamethyldisiloxane | 86.7% | by weight |
| 2-Propanol, ultra-high purity | 5% | by weight |
| Phospholipid | 8% | by weight |
| Peppermint oil | 0.2% | by weight |

When the anhydrous sprayable composition described above was applied to the skin, it spontaneously developed liposomes which had an average particle size in the range of 205 nm.

### EXAMPLE 10

A sprayable liquid composition was prepared from the following ingredients for treatment of dermatomycoses caused by dermatophytes, yeasts (e.g., Candida species), molds and other fungi and involving inflammatory and/or eczematous skin manifestations and/or itching:

| | | |
|---|---|---|
| Clotrimazole | 0.8% | by weight |
| 2-Propanol | 11.2% | by weight |
| Hexamethyldisiloxane | 77.8% | by weight |
| Phospholipid | 10% | by weight |
| Peppermint oil | 0.2% | by weight |

When the anhydrous sprayable composition described above was applied to the skin, it spontaneously developed liposomes which had an average particle size in the range of 170 nm.

### EXAMPLE 11

A sprayable liquid composition for treatment of sports injuries, accident injuries, soft tissue rheumatism, pain involving the motor system in the joints, muscles and/or tendons caused by various accidents, strains or other disease was prepared from the following ingredients:

| | | |
|---|---|---|
| Dexibuprofen | 8.3% | by weight |
| 2-Propanol | 9% | by weight |
| Hexamethyldisiloxane | 70.2% | by weight |
| Phospholipid | 12.3% | by weight |
| Peppermint oil | 0.2% | by weight |

When the anhydrous sprayable composition described above was applied to the skin, it would spontaneously develop liposomes which had an average particle size in the range of 105 nm.

### EXAMPLE 12

A sprayable liquid composition for treatment of sports injuries, accident injuries, soft tissue rheumatism, pain involving the motor system in the joints, muscles and/or tendons caused by various accidents, strains or other disease was prepared from the following ingredients:

| | | |
|---|---|---|
| Ibuprofen | 8.3% | by weight |
| 2-Propanol | 9% | by weight |
| Hexamethyldisiloxane | 70.2% | by weight |
| Phospholipid | 12.3% | by weight |
| Peppermint oil | 0.2% | by weight |

When the anhydrous sprayable composition described above was applied to the skin, it spontaneously developed liposomes which had an average particle size in the range of 135 nm.

### EXAMPLE 13

A sprayable liquid composition for treatment of endogenous eczema (atopic dermatitis, neurodermatitis), degenerative dyshidrotic eczema vulgaris, contact eczema and neurodermatitis was prepared from the following ingredients:

| | | |
|---|---|---|
| Prednisolone | 0.1% | by weight |
| 2-Propanol | 5% | by weight |
| Hexamethyldisiloxane | 86.7% | by weight |
| Phospholipid | 8% | by weight |
| Peppermint oil | 0.2% | by weight |

When the anhydrous sprayable composition described above was applied to skin, it spontaneously developed liposomes which had an average particle size in the range of 400 nm.

### EXAMPLE 14

A sprayable liquid composition for treatment of acute and chronic eczema, atopic dermatitis (neurodermatitis), psoriasis and first-degree burns was prepared from the following ingredients:

| | | |
|---|---|---|
| Prednicarbate | 0.1% | by weight |
| 2-Propanol | 5% | by weight |
| Hexamethyldisiloxane | 86.7% | by weight |
| Phospholipid | 8% | by weight |
| Peppermint oil | 0.2% | by weight |

When the anhydrous sprayable composition described above was applied to the skin, it spontaneously developed liposomes which had an average particle size in the range of 295 nm.

### EXAMPLE 15

A sprayable liquid composition for treatment of acute and chronic eczema, atopic dermatitis (neurodermatitis), psoriasis, first degree burns was prepared from the following ingredients:

| | | |
|---|---|---|
| Prednicarbate | 0.1% | by weight |
| 2-Propanol | 5% | by weight |
| Octamethylcyclotetrasiloxane | 74.7% | by weight |
| Phospholipid | 20% | by weight |
| Peppermint oil | 0.2% | by weight |

When the anhydrous sprayable composition described above was applied to the skin, it spontaneously developed liposomes which had an average particle size in the range of 412 nm.

### EXAMPLE 16

A sprayable liquid composition for treatment of sports injuries, accident injuries, soft tissue rheumatism, pain involving the motor system in the joints, muscles and/or tendons caused by various accidents, strains or other disease was prepared from the following ingredients:

| | | |
|---|---|---|
| Diclofenac | 5% | by weight |
| 2-Propanol | 9% | by weight |
| Hexamethyldisiloxane | 72.5% | by weight |
| Phospholipid | 12.3% | by weight |
| Peppermint oil | 0.2% | by weight |

When the anhydrous sprayable composition described above was applied to the skin, it spontaneously developed liposomes which had an average particle size in the range of 50 to 400 nm.

### EXAMPLE 17

A sprayable liquid composition for treatment of fungal diseases was prepared from the following ingredients:

| | | |
|---|---|---|
| Clotrimazole | 1% | by weight |
| 2-Propanol | 11% | by weight |
| Hexamethyldisiloxane | 79.8% | by weight |
| Phospholipid | 8% | by weight |
| Peppermint oil | 0.2% | by weight |

When the anhydrous sprayable composition described above was applied to the skin, it spontaneously developed liposomes which had an average particle size in the range of 230 nm.

The stability of this composition according to example 17 was measured. After extreme storage for six months at 40° C., the clotrimazole content of the composition stored in this way was found to be 99.9%, based on the initial concentration, so that only 0.1% of the active ingredient used originally had been degraded to carbinol.

The phospholipids used in examples 1 through 3 and 7 through 12 each contained 25% by weight ethanol and 75% by weight phospholipid which in turn contained 76±3% by weight phosphatidylcholine, 3±3% by weight lysophosphatidylcholine, up to 8% by weight phosphatidic acid, up to 4% by weight phosphatidylethanolamine and a maximum of 9% by weight other phospholipid and non-phospholipid ingredients, such as in particular oils, fats and/or triglycerides.

The phospholipid used in examples 13 and 14 contained 15% by weight isopropanol and 85% by weight phospholipid which in turn contained 76±3% by weight phosphatidylcholine, 3±3% by weight lysophosphatidylcholine, up to 8% by weight phosphatidic acid, up to 4% by weight phosphatidylethanolamine and a maximum of 9% by weight other phospholipid and non-phospholipid ingredients, such as in particular oils, fats and/or triglycerides.

The phospholipid used in examples 15 through 17 contained 10% by weight ethanol and 90% by weight phospholipid, which in turn contained 45±5% by weight phosphatidylcholine, 15±3% by weight phosphatidylethanolamine, 25±4% by weight phosphatidylinositol and a maximum of 15% by weight other phospholipid and non-phospholipid ingredients, such as in particular oils, fats and/or triglycerides.

The procedure followed to prepare the compositions described in example 10 was the same as that described in example 1 above.

To produce the composition described in examples 2 through 9 and 11 through 17, the respective active ingredient was taken first and dissolved while stirring at room temperature in a phase containing the phospholipid plus optionally the additional ingredients (alcohol, emulsifier, scents, etc.). After a complete and transparent solution had been prepared, the siloxane was added while continuing to stir at room temperature.

To prepare the composition described in examples 1 and 10, the respective active ingredient was taken first and while stirring, was dissolved at room temperature in a phase containing the phospholipid, optionally the other ingredients (alcohol, emulsifier, scents, etc.) and between 10% by weight and 40% by weight of the respective total amount of the siloxane to be used. After preparing a complete and transparent solution, the remaining amount of the siloxane was added while continuing to stir at room temperature.

1. A pharmaceutical and/or cosmetic composition for use on humans, animals or plants, comprising at least one pharmaceutical and/or cosmetic active ingredient, at least one phospholipid and at least one organosilicon compound on the bases of a diorganosiloxane, characterized in that the composition contains hexamethyldisiloxane as organosilicon compound.

2. The composition according to claim 1, characterized in that the composition comprises as the phospholipid such a phospholipid, being isolated from natural starting materials.

3. The composition according to claim 1 or 2, characterized in that the composition comprises as the phospholipid at least one phospholipid having a high phosphatidylcholine content, preferably such a phospholipid having more than 70% by weight phosphatidylcholine.

4. The composition according to claim 3, characterized in that the phospholipid contains at least 80% by weight and preferably more than 90% by weight phosphatidylcholine.

5. The composition according to one of the preceding claims, characterized in that the phospholipid is a hydrogenated phospholipid, preferably a hydrogenated phosphatidylcholine.

6. The composition according to one of the preceding claims, characterized in that the composition comprises the hexamethyldisiloxane in a concentration between 5% by weight and 96% by weight, based on the total weight of the composition, and at least one phospholipid in a concentration between 2% by weight and 30% by weight, based on the total weight of the composition.

7. The composition according to claim 6, characterized in that the composition contains between 20% by weight and 85% by weight of the hexamethyldisiloxane and between 5% by weight and 20% by weight of the at least one phospholipid.

8. The composition according to one of the preceding claims, characterized in that the composition comprises between 0% by weight and 5% by weight water, and the hexamethyldisiloxane and the phospholipid contained in the composition are mixed together so that the composition forms liposomes and/or micelles spontaneously on coming in contact with water.

9. The composition according to claim 8, characterized in that the liposomes and/or micelles formed spontaneously by adding water have an average particle size between 50 nm and 4000 nm, in particular between 250 nm and 1500 nm.

10. The composition according to one of the preceding claims, characterized in that the composition also comprises water, alcohol, antioxidants, non-phospholipid emulsifiers, pharmaceutical active ingredients, cosmetic active ingredients and/or other auxiliary substances, in addition to the at least one organosilicon compound and the at least one phospholipid.

11. The composition according to claim 10, characterized in that the concentration of the at least one pharmaceutical active ingredient and/or the at least one cosmetic active ingredient in the composition varies between 0.001% by weight and 30% by weight, in particular between 0.1% by weight and 15% by weight, based on the ready-to-use composition.

12. The composition according to one of the preceding claims, characterized in that the composition also contains at least one alcohol, where the concentration of the at least one alcohol amounts to a maximum of 14% by weight and preferably varies between 0.1% by weight and 8.5% by weight, each based on the ready-to-use composition.

13. The composition according to one of the preceding claims, characterized in that the composition for use on humans or animals comprises at least one cosmetic and/or pharmaceutical active ingredient which is selected from the group consisting of local anesthetics, antiallergics, dermatologics, active ingredients to combat influenza infections and colds, active ingredients for treatment of neuropathies, active ingredients for treatment of circulation disorders, chemotherapeutics, quinine, antimycotics, antibiotics, thalidomide, analgesics, non-steroidal anti-inflammatory drugs, leukotrienes, leukotriene inhibitors, antiandrogens, corticosteroids, opiate receptor antagonists, blood coagulation inhibitors, platelet aggregation inhibitors, histamine antagonists, regulatory and enzymatic peptides and proteins, nucleic acids and oligopeptides, antidiabetics, prostaglandins, prostaglandin synthesis inhibitors, antiviral or virustatic substances, antimicrobial substances, active ingredients against prions, immunosuppressants, hormones, active ingredients for treatment of wounds and especially chronic wounds, vitamins, plant extracts or extracts of plant extracts, psychopharmaceuticals, active ingredients to influence sleep, analeptics, general anesthetics, muscle relaxants, antiepileptics, antiparkinsonian drugs, antiemetics, substances that act on the ganglia, substances that act on the sympathetic nervous system, substances that act on the parasympathetic nervous system, calcium antagonists, cardiovascular agents, antiasthmatics, antitussives, expectorants, hepatics, diuretics, choleretics, disinfectants, trace elements, anti-infectives, cytostatics, antimetabolites, hormone antagonists and/or immunomodulators.

14. The composition according to one of the claims 1 to 12, characterized in that the composition for use on plants contains as the active ingredient a fungicide, an insecticide, a herbicide and/or a fertilizer absorbable through leaf parts and/or above-ground plant parts.

15. The composition according to one of the preceding claims, characterized in that the composition has a liquid to pasty viscosity for topical application, whereby the viscosity of the composition preferably varies between 0.4 cSt and 10,000 cSt, in particular between 0.6 cSt and 1500 cSt.

16. The composition according to claim 15, characterized in that the composition comprises the hexamethyldisiloxane and the at least one phospholipid in such a coordinated weight ratio that after applying the composition to human or animal skin in a concentration between 0.05 and 0.3 g per 100 cm² skin, no visible residues of the composition remain on the skin within a period of 1 second to 60 seconds, preferably within a period of 3 seconds to 20 seconds.

17. The composition according to one of the preceding claims, characterized in that the composition comprises at least one vitamin as the pharmaceutical and/or cosmetic active ingredient.

18. The composition according to claim 17, characterized in that the vitamin is vitamin C, vitamin E, vitamin A and/or pro-vitamin A.

19. The composition according to one of the preceding claims, characterized in that the composition is in such a form of administration which permits administration of the composition as a capsule, suppository, plaster, spray, mist, gel, powder and/or cream.

20. The composition according to one of the preceding claims, characterized in that it is prepared for topical application of pharmaceutical and/or cosmetic active ingredients, for buccal application of these active ingredients and/or for oral administration of these active ingredients.
